(19) United States
(12) Patent Application Publication
(10) Pub. No.: US 2017/0274110 A1
(43) Pub. Date: Sep. 28, 2017

(54) LONG LASTING FRESHENING PRODUCTS AND METHOD OF FRESHENING THE AIR

(71) Applicant: The Procter & Gamble Company, Cincinnati, OH (US)

(72) Inventors: Chisomaga Ugochi NWACHUKWU, Cincinnati, OH (US); Judith Ann HOLLINGSHEAD, Batavia, OH (US); Zaiyou LIU, West Chester, OH (US); George Kavin MORGAN, III, Hamilton, OH (US); Christine Marie READNOUR, Ft. Mitchell, KY (US); Laura Jane ZIELEWICZ, Cincinnati, OH (US)

(21) Appl. No.: 15/469,615

(22) Filed: Mar. 27, 2017

Related U.S. Application Data
(60) Provisional application No. 62/313,831, filed on Mar. 28, 2016.

(51) Int. Cl. A61L 9/14; A61L 9/04; A61L 9/01

(52) U.S. Cl. CPC A61L 9/14; A61L 9/01; A61L 9/04

(57) ABSTRACT

A freshening product is provided. The freshening product includes a spray dispenser containing a freshening composition and a propellant. The freshening composition includes about 0.02 wt. % to about 1.0 wt. %, based on the weight of the composition, of a sulfur-containing pro-perfume; a perfume mixture comprising at least one perfume raw material; and a carrier. The freshening composition comprises a weight ratio of perfume mixture to sulfur-containing pro-perfume of about 6:1 to about 35:1, by weight of the freshening composition.

### FIELD

The present disclosure relates to long-lasting freshening products and methods of freshening the air, and, more particularly, to freshening compositions that comprise sulfur containing pro-perfumes and products and methods of freshening the air with the same.

### BACKGROUND

Freshening compositions for reducing or masking malodors with scent on inanimate surfaces such as fabrics and in air are currently available and are described in the patent literature. Compositions that are capable of delivering long-lasting scent on inanimate surfaces and in the air also exist. Some compositions contain high amounts of perfume in order to provide long-lasting scent or freshness into the air. However, such compositions may deliver an overwhelming amount of scent into the air after the product is initially delivered to the air. As such, it would be beneficial to provide a freshening composition that provides long-lasting freshness to the air while delivering a relatively consistent scent over time.

### SUMMARY

A. A freshening product comprising a spray dispenser, the spray dispenser containing a freshening composition and a propellant, the freshening composition comprising: about 0.02 wt. % to about 1.0 wt. %, based on the weight of the composition, of a sulfur-containing pro-perfume; a perfume mixture comprising at least one perfume raw material; and a carrier, wherein the freshening composition comprises a weight ratio of perfume mixture to sulfur-containing pro-perfume of about 6:1 to about 35:1, by weight of the freshening composition.
B. The freshening product of Paragraph A, wherein the freshening composition comprises a pH in the range of about 3 to about 9, more preferably in the range of about 4 to about 6.5.
C. The freshening product according to any of Paragraphs A-B, wherein the weight ratio of perfume mixture to sulfur-containing pro-perfume is about 8:1 to about 25:1, more preferably about 10:1 to about 20:1, by weight of the composition.
D. The freshening product according to any of Paragraphs A-C, wherein the sulfur-containing pro-perfume is present at a level of about 0.02 wt. % to about 1.0 wt. %, preferably about 0.02 wt. % to about 0.8 wt. %, more preferably about 0.03 wt. % to about 0.3 wt. %, most preferably about 0.03 wt % to about 0.09 wt %, by weight of the freshening composition.
E. The freshening product according to any of Paragraphs A-D, wherein the perfume mixture is present at a level of about 0.2 wt. % to about 1.4 wt. %, by weight of the freshening composition.
F. The freshening product according to any of Paragraphs A-E, wherein the freshening composition further comprises a carboxylic acid, more preferably the carboxylic acid is citric acid or polyacrylic acid.
G. The freshening product according to any of Paragraphs A-F, wherein the freshening composition comprises a polyol.
H. The freshening product according to any of Paragraphs A-G, wherein the perfume mixture is present at a level of about 0.2 wt. % to about 2.0 wt. %, by weight of the freshening composition.
I. The freshening product according to any of Paragraphs A-H, wherein the perfume mixture comprises at least one material selected from the group consisting of: 3-(1,3-Benzodioxol-5-yl)-2-methylpropanal, canthoxal, vanillin, ethyl vanillin, beta ionone, dimethyl anthranilate, citral, and combinations thereof.
J. The freshening product according to any of Paragraphs A-I, wherein the sulfur-containing pro-perfume is a C4-C12 thio-damascone.
K. The freshening product according to any of Paragraphs A-J, wherein the spray dispenser is plastic.
L. The freshening product according to any of Paragraphs A-K, wherein the spray dispenser is transparent or transluscent.
M. The freshening product of Paragraphs A-L, wherein the propellant comprises compressed gas or a hydrocarbon.
N. The freshening product according to any of Paragraphs A-M further comprising a solubilizer.
O. The freshening product according to any of Paragraphs A-N further comprising a wetting agent.
P. The freshening product according to any of Paragraphs A-O, wherein the freshening composition further comprises a secondary or tertiary amine.
Q. The freshening product according to any of Paragraphs A-P, wherein the freshening composition is an aqueous composition.
R. A method of freshening the air, the method comprising the step of: providing a freshening composition in a pressurized spray dispenser, the freshening composition comprising: about 0.02 wt. % to about 1.0 wt. %, based on the weight of the composition, of a sulfur-containing pro-perfume; a perfume mixture comprising at least one perfume raw material; and a carrier, wherein the composition comprises a ratio of perfume mixture to sulfur-containing pro-perfume of about 6:1 to about 35:1; and spraying the freshening composition into the air in the form of spray droplets.
S. The method of Paragraph R, wherein the composition comprises a pH in the range of about 3 to about 9, more preferably a range of about 4 to about 6.5.
T. The method according to any of Paragraphs R-S, wherein the weight ratio of perfume mixture to sulfur-containing pro-perfume is about 8:1 to about 25:1, more preferably about 10:1 to about 20:1, by weight of the composition.
U. The method according to any of Paragraphs R-T, wherein the sulfur-containing pro-perfume is present at a level of about 0.02 wt. % to about 1.0 wt. %, preferably about 0.02 wt. % to about 0.8 wt. %, more preferably about 0.03 wt. % to about 0.3 wt. %, most preferably about 0.03 wt % to about 0.09 wt %, by weight of the freshening composition.
V. The method according to any of Paragraphs R-U wherein the perfume mixture is present at a level of about 0.2 wt. % to about 1.4 wt. %, by weight of the freshening composition.
W. The method according to any of Paragraphs R-V further comprising a carboxylic acid, preferably the carboxylic acid is citric acid or polyacrylic acid.
X. The method according to any of Paragraphs R-W, wherein the perfume mixture is from about 0.2 wt. % to about 2.0 wt. %, by weight of the composition.
Y. The method according to any of Paragraphs R-X, wherein the perfume mixture comprises at least one material selected from the group consisting of: 3-(1,3-Benzodioxol-5-yl)-2-methylpropanal, canthoxal, vanillin, ethyl vanillin, beta ionone, dimethyl anthranilate, citral, and combinations thereof.
Z. The method according to any of Paragraphs R-Y, wherein the sulfur-containing pro-perfume is a C4-C12 thio-damascone.
AA. The method according to any of Paragraphs R-Z, wherein the spray dispenser is plastic.
BB. The method according to any of Paragraphs R-AA, wherein the spray dispenser is transparent or translucent.
CC. The method according to any of Paragraphs R-BB, wherein the propellant comprises compressed gas or a hydrocarbon.
DD. The method according to any of Paragraphs R-CC further comprising a solubilizer.
EE. The method according to any of Paragraphs R-DD further comprising a wetting agent.
FF. The method according to any of Paragraphs R-EE further comprising a primary or tertiary amine.
GG. The method according to any of Paragraphs R-FF, wherein the spray droplets have a mean particle size from about 10 microns to about 100 microns.
HH. The method according to any of Paragraphs R-GG, wherein the step of spraying the freshening composition further comprises spraying the freshening composition at a flow rate in the range of about 0.1 g/s to about 2.5 g/s.
II. The method according to any of Paragraphs R-HH, wherein the freshening composition comprises a polyol.
JJ. The method according to any of Paragraphs R-II, wherein the freshening composition is an aqueous composition.

### DETAILED DESCRIPTION

The freshening composition of the present disclosure is designed to extend scent release into the air or onto an inanimate surface. Such prolonged freshness and malodor reduction may last for at least about four hours, or at least about six hours, or at least about eight hours, or at least about 24 hours, or at least about 48 hours after treating a space with the freshening composition. The freshening compositions of the present disclosure may also deliver relatively consistent scent over an extended period of time. Moreover, the freshening compositions of the present disclosure may be designed to be stable over an extended shelf-life.

The freshening compositions of the present disclosure include a sulfur-containing pro-perfume, perfume raw material(s), and a carrier.

### Sulfur-Containing Pro-Perfume

The term “sulfur-containing pro-perfume” herein refers to a type of pro-perfume compound that contains sulfur. The term “pro-perfume” herein refers to compounds resulting from the reaction of perfume raw materials (“PRMs” or, singularly, “PRM”) with other chemicals, which have a covalent bond between one or more PRMs and these other chemicals. The PRM is converted into a new material called a pro-perfume compound, which then may release the original PRM (i.e., pre-converted) upon exposure to a trigger such as water or light or atmospheric oxygen. Suitable pro-perfume compounds and methods of making the same can be found in U.S. Pat. Nos. 7,018,978; 6,861,402; 6,544,945; 6,093,691; 6,165,953; and 6,096,918.

The sulfur-containing pro-perfume herein may comprise a compound of formula (I):

Y-S-G-Q (I)

wherein:
(i) Y is a radical selected from the group consisting of (Y-1) to (Y-7) shown herein below, including isomeric forms:

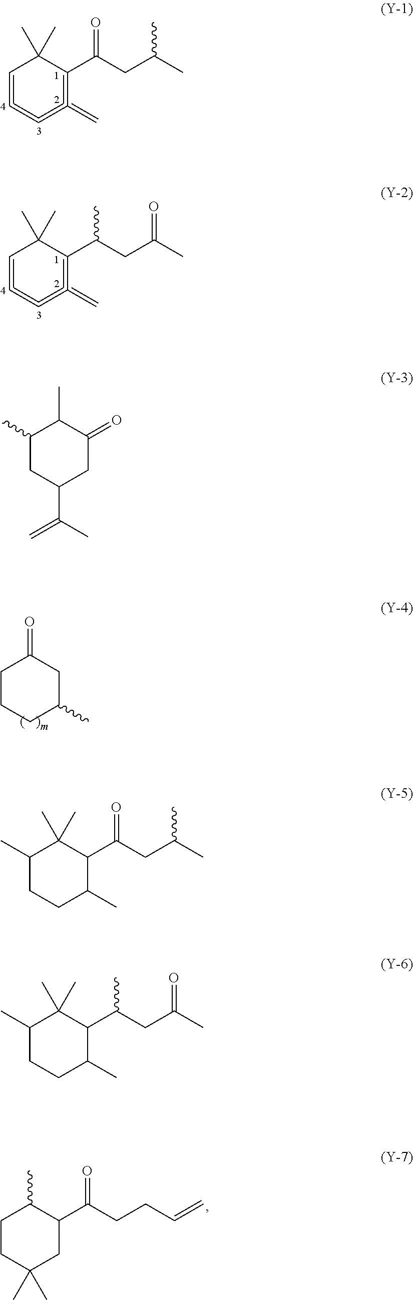

wherein the wavy lines represent the location of the sulfur (S) bond, and the dotted lines represent a single or double bond;
(ii) G is selected from a divalent or trivalent radical derived from a linear or branched alkyl or alkenyl radical having from 2 to 15 carbon atoms; and
(iii) Q is selected from a hydrogen, a —S—Y group, or a —NR²—Y group, wherein Y is independently selected as defined above, and R² is selected from a hydrogen or a C₁-C₃ alkyl group.

G may be a divalent or trivalent radical, preferably a divalent radical derived from a linear or branched alkyl or alkenyl radical having from 2 to 15 carbon atoms, substituted with one or more groups selected from the group consisting of —OR¹, —NR¹₂, —COOR¹, R¹ groups, and a combination thereof, wherein R¹ is selected from a hydrogen or a C₁ to C₆ alkyl or alkenyl group. Preferably, G is a divalent radical derived from a linear or branched alkyl or alkenyl radical having from 2 to 15 carbon atoms, substituted with at least one —COOR¹ group, preferably substituted with a —COOR¹ group, wherein R¹ is selected from a hydrogen or a C₁ to C₆ alkyl or alkenyl group. Even more preferably, G is a divalent radical derived from a linear alkyl radical having a —CH₂CH(COOR¹) group, wherein R¹ is a hydrogen or a methyl or ethyl group. G may be a divalent radical derived from a linear alkyl radical having from 8 to 15 carbon atoms which is either substituted or un-substituted.

The sulfur-containing pro-perfume may be a compound of formula (I) wherein Y is selected from Y-1, Y-2 or Y-3 groups as defined above, and G and Q are defined in any one of the above-described examples. The sulfur-containing pro-perfume may be a sulfide.

Preferably, the sulfur-containing pro-perfume is selected from the group consisting of methyl or ethyl 2-(4-oxo-4-(2,6,6-trimethylcyclohex-3-en-1-yl)butan-2-ylamino)-3-(4-oxo-4-(2,6,6-trimethylcyclohex-3-en-1-yl)butan-2-ylthio)propanate, methyl or ethyl 2-(4-oxo-4-(2,6,6-trimethylcyclohex-2-en-1-yl)butan-2-ylamino)-3-(4-oxo-4-(2,6,6-trimethylcyclohex-2-en-1-yl)butan-2-ylthio)propanate, methyl or ethyl 2-(2-oxo-4-(2,6,6-trimethylcyclohex-1-en-1-yl)butan-4-ylamino)-3-(2-oxo-4-(2,6,6-trimethylcyclohex-1-en-1-yl)butan-4-ylthio)propanate, methyl or ethyl 2-(2-oxo-4-(2,6,6-trimethylcyclohex-2-en-1-yl)butan-4-ylamino)-3-(2-oxo-4-(2,6,6-trimethylcyclohex-2-en-1-yl)butan-4-ylthio)propanate, 3-(dodecylthio)-1-(2,6,6-trimethylcyclohex-3-en-1-yl)-1-butanone, 3-(dodecylthio)-1-(2,6,6-trimethylcyclohex-2-en-1-yl)-1-butanone, 4-(dodecylthio)-4-(2,6,6-trimethylcyclohex-2-en-1-yl)-2-butanone, 4-(dodecylthio)-4-(2,6,6-trimethylcyclohex-1-en-1-yl)-2-butanone, 2-dodecylsulfanyl-5-methyl-heptan-4-one, 2-cyclohexyl-1-dodecylsulfanyl-hept-6-en-3-one, 3-(dodecylthio)-5-isopropenyl-2-methylcyclohexanone, and a combination thereof.

More preferably, the sulfur-containing pro-perfume compound is selected from the group consisting of 3-(dodecylthio)-1-(2,6,6-trimethylcyclohex-3-en-1-yl)-1-butanone, 4-(dodecylthio)-4-(2,6,6-trimethylcyclohex-2-enl-yl)-2-butanone, 4-(dodecylthio)-4-(2,6,6-trimethylcyclohex-1-en-1-yl)-2-butanone and 3-(dodecylthio)-5-isopropenyl-2-methylcyclohexanone, and a combination thereof. 3-(dodecylthio)-1-(2,6,6-trimethylcyclohex-3-en-1-yl)-1-butanone is the most preferred sulfur-containing pro-perfume compound, such as Haloscent® D available from Firmenich located in Geneva, Switzerland.

The sulfur-containing pro-perfume compound may be present at various levels in the composition. Preferably, the sulfur-containing pro-perfume compound is present in an amount from about 0.01% to about 1.0%, alternatively from about 0.02% to about 0.8%, alternatively from about 0.03% to about 0.5%, alternatively about 0.03% to about 0.3%, alternatively about 0.03% to about 0.09%, alternatively at least about 0.02%, alternatively at least about 0.03%, alternatively at least about 0.04%, alternatively at least about 0.05%, by weight of the composition.

The freshening composition may comprise dodecyl thio-damascone having the general structure shown below.

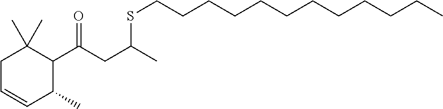 [SMILES: CCCCCCCCCCCCSC(C)CC(=O)C1[C@H](C)C=CCC1(C)C]

Thio-damascone may be present in an amount from about 0.01% to about 1.0%, alternatively from about 0.02% to about 0.8%, alternatively from about 0.03% to about 0.5%, alternatively about 0.03% to about 0.3%, alternatively about 0.03% to about 0.09%, alternatively at least about 0.02%, alternatively at least about 0.03%, alternatively at least about 0.04%, alternatively at least about 0.05%, by weight of the composition.

### Perfume Mixture

The freshening composition also includes a perfume mixture comprising at least one perfume raw materials (PRMs). Various PRMs may be used. The freshening composition may include a perfume mixture comprising one or more of the following perfume raw materials. As used herein, a “perfume raw material” refers to one or more of the following ingredients: fragrant essential oils; aroma compounds; pro-perfumes; materials supplied with the fragrance essential oils, aroma compounds, and/or pro-perfumes, including stabilizers, diluents, processing agents, and contaminants; and any material that commonly accompanies fragrant essential oils, aroma compounds, and/or pro-perfumes.

The PRM may comprise a ketone. The ketone can comprise any PRMs which contain one or more ketone moieties and which can impart a desirable scent. The PRM may comprise ketone comprising a PRM selected from the group consisting of buccoxime; iso jasmone; methyl beta naphthyl ketone; musk indanone; tonalid/musk plus; alpha-damascone, beta-damascone, delta-damascone, iso-damascone, damascenone, damarose, methyl-dihydrojasmonate, menthone, carvone, camphor, fenchone, alpha-ionone, beta-ionone, dihydro-beta-ionone, gamma-methyl so-called ionone, fleuramone, dihydrojasmone, cis-jasmone, iso-e-super, methyl-cedrenyl-ketone or methyl-cedrylone, acetophenone, methyl-acetophenone, para-methoxy-acetophenone, methyl-beta-naphtyl-ketone, benzyl-acetone, benzophenone, para-hydroxy-phenyl-butanone, celery ketone or livescone, 6-isopropyldecahydro-2-naphtone, dimethyl-octenone, freskomenthe, 4-(1-ethoxyvinyl)-3,3,5,5,-tetramethyl-cyclohexanone, methyl-heptenone, 2-(2-(4-methyl-3-cyclohexen-1-yl)propyl)-cyclopentanone, 1-(p-menthen-6(2)-yl)-1-propanone, 4-(4-hydroxy-3-methoxyphenyl)-2-butanone, 2-acetyl-3,3-dimethyl-norbornane, 6,7-dihydro-1,1,2,3,3-pentamethyl-4(5h)-indanone, 4-damascol, dulcinyl or cassione, gelsone, hexalon, isocyclemone e, methyl cyclocitrone, methyl-lavender-ketone, orivon, para-tertiary-butyl-cyclohexanone, verdone, delphone, muscone, neobutenone, plicatone, veloutone, 2,4,4,7-tetramethyl-oct-6-en-3-one, tetrameran, hedione, floralozone, gamma undecalactone, ethylene bras sylate, pentadecanolide, methyl nonyl ketone, cyclopentadecanone, cyclic ethylene dodecanedioate, 3,4,5,6-tetrahydropseudoionone, 8-hexadecenolide, dihydrojasmone, 5-cyclohexadecenone, and a combination thereof.

The PRM comprising ketone comprises a PRM selected from the group consisting of alpha-damascone, delta-damascone, iso-damascone, carvone, gamma-methyl-ionone, beta-ionone, iso-e-super, 2,4,4,7-tetramethyl-oct-6-en-3-one, benzyl acetone, beta-damascone, damascenone, methyl dihydrojasmonate, methyl cedrylone, hedione, floralozone, and a combination thereof. Preferably, the PRM comprising ketone comprises delta-damascone.

The freshening composition may include a mixture of aldehydes that contribute to scent character and neutralize malodors in vapor and/or liquid phase via chemical reactions. Aldehydes that are partially reactive or volatile may be considered a reactive aldehyde as used herein. Reactive aldehydes may react with amine-based odors, following the path of Schiff-base formation. Reactive aldehydes may also react with sulfur-based odors, forming thiol acetals, hemi thiolacetals, and thiol esters in vapor and/or liquid phase. It may be desirable for these vapor and/or liquid phase reactive aldehydes to have virtually no negative impact on the desired perfume character, color or stability of a product.

The freshening composition may include a mixture of aldehydes that are partially volatile which may be considered a volatile aldehyde as used herein. The volatile aldehydes may also have a certain boiling point (B.P.) and octanol/water partition coefficient (P). The boiling point referred to herein is measured under normal standard pressure of 760 mmHg. The boiling points of many volatile aldehydes, at standard 760 mm Hg are given in, for example, “Perfume and Flavor Chemicals (Aroma Chemicals),” written and published by Steffen Arctander, 1969.

The octanol/water partition coefficient of a volatile aldehyde is the ratio between its equilibrium concentrations in octanol and in water. The partition coefficients of the volatile aldehydes used in the malodor control composition may be more conveniently given in the form of their logarithm to the base 10, log P. The log P values of many volatile aldehydes have been reported. See, e.g., the Pomona92 database, available from Daylight Chemical Information Systems, Inc. (Daylight CIS), Irvine, Calif. However, the log P values are most conveniently calculated by the “CLOGP” program, also available from Daylight CIS. This program also lists experimental log P values when they are available in the Pomona92 database. The “calculated log P” (Clog P) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990). The fragment approach is based on the chemical structure of each volatile aldehyde, and takes into account the numbers and types of atoms, the atom connectivity, and chemical bonding. The Clog P values, which are the most reliable and widely used estimates for this physicochemical property, are preferably used instead of the experimental log P values in the selection of volatile aldehydes for the malodor control composition.

The Clog P values may be defined by four groups and the volatile aldehydes may be selected from one or more of these groups. The first group comprises volatile aldehydes that have a B.P. of about 250° C. or less and Clog P of about 3 or less. The second group comprises volatile aldehydes that have a B.P. of 250° C. or less and Clog P of 3.0 or more. The third group comprises volatile aldehydes that have a B.P. of 250° C. or more and Clog P of 3.0 or less. The fourth group comprises volatile aldehydes that have a B.P. of 250° C. or more and Clog P of 3.0 or more. The malodor control composition may comprise any combination of volatile aldehydes from one or more of the Clog P groups.

The malodor control composition may comprises, by total weight of the freshening composition, from about 0% to about 30% of volatile aldehydes from group 1, alternatively about 25%; and/or about 0% to about 10% of volatile aldehydes from group 2, alternatively about 10%; and/or from about 10% to about 30% of volatile aldehydes from group 3, alternatively about 30%; and/or from about 35% to about 60% of volatile aldehydes from group 4, alternatively about 35%.

Exemplary reactive and/or volatile aldehydes which may be used in a freshening composition include, but are not limited to, Adoxal (2,6,10-Trimethyl-9-undecenal), Bourgeonal (4-t-butylbenzenepropionaldehyde), Lilestralis 33 (2-methyl-4-t-butylphenyl)propanal), Cinnamic aldehyde, cinnamaldehyde (phenyl propenal, 3-phenyl-2-propenal), Citral, Geranial, Neral (dimethyloctadienal, 3,7-dimethyl-2,6-octadien-1-al), Cyclal C (2,4-dimethyl-3-cyclohexen-1-carbaldehyde), Florhydral (3-(3-Isopropyl-phenyl)-butyraldehyde), Citronellal (3,7-dimethyl 6-octenal), Cymal, cyclamen aldehyde, Cyclosal, Lime aldehyde (Alpha-methyl-p-isopropyl phenyl propyl aldehyde), Methyl Nonyl Acetaldehyde, aldehyde C12 MNA (2-methyl-1-undecanal), Hydroxycitronellal, citronellal hydrate (7-hydroxy-3,7-dimethyl octan-1-al), Helional (3-(1,3-Benzodioxol-5-yl)-2-methylpropanal; 2-Methyl-3-(3,4-methylenedioxyphenyl)propanal), Intreleven aldehyde (undec-10-en-1-al), Ligustral, Trivertal (2,4-dimethyl-3-cyclohexene-1-carboxaldehyde), Jasmorange, satinaldehyde (2-methyl-3-tolylproionaldehyde, 4-dimethylbenzenepropanal), Lyral (4-(4-hydroxy-4-methyl pentyl)-3-cyclohexene-1-carboxaldehyde), Melonal (2,6-Dimethyl-5-Heptenal), Methoxy Melonal (6-methoxy-2,6-dimethylheptanal), methoxycinnamaldehyde (trans-4-methoxycinnamaldehyde), Myrac aldehyde isohexenyl cyclohexenyl-carboxaldehyde, trifernal ((3-methyl-4-phenyl propanal, 3-phenyl butanal), lilial, P.T. Bucinal, lysmeral, benzenepropanal (4-tert-butyl-alpha-methyl-hydrocinnamaldehyde), Dupical, tricyclodecylidenebutanal (4-Tricyclo5210-2,6decylidene-8butanal), Melafleur (1,2,3,4,5,6,7,8-octahydro-8,8-dimethyl-2-naphthaldehyde), Methyl Octyl Acetaldehyde, aldehyde C-11 MOA (2-mehtyl deca-1-al), Onicidal (2,6,10-trimethyl-5,9-undecadien-1-al), Citronellyl oxyacetaldehyde, Muguet aldehyde 50 (3,7-dimethyl-6-octenyl) oxyacetaldehyde), phenylacetaldehyde, Mefranal (3-methyl-5-phenyl pentanal), Triplal, Vertocitral dimethyl tetrahydrobenzene aldehyde (2,4-dimethyl-3-cyclohexene-1-carboxaldehyde), 2-phenylproprionaldehyde, Hydrotropaldehyde, Canthoxal, anisylpropanal 4-methoxy-alpha-methyl benzenepropanal (2-anisylidene propanal), Cylcemone A (1,2,3,4,5,6,7,8-octahydro-8,8-dimethyl-2-naphthaldehyde), and Precylcemone B (1-cyclohexene-1-carboxaldehyde).

Still other exemplary aldehydes include, but are not limited to, acetaldehyde (ethanal), pentanal, valeraldehyde, amylaldehyde, Scentenal (octahydro-5-methoxy-4,7-Methano-1H-indene-2-carboxaldehyde), propionaldehyde (propanal), Cyclocitral, beta-cyclocitral, (2,6,6-trimethyl-1-cyclohexene-1-acetaldehyde), Iso Cyclocitral (2,4,6-trimethyl-3-cyclohexene-1-carboxaldehyde), isobutyraldehyde, butyraldehyde, isovaleraldehyde (3-methyl butyraldehyde), methylbutyraldehyde (2-methyl butyraldehyde, 2-methyl butanal), Dihydrocitronellal (3,7-dimethyl octan-1-al), 2-Ethylbutyraldehyde, 3-Methyl-2-butenal, 2-Methylpentanal, 2-Methyl Valeraldehyde, Hexenal (2-hexenal, trans-2-hexenal), Heptanal, Octanal, Nonanal, Decanal, Lauric aldehyde, Tridecanal, 2-Dodecanal, Methylthiobutanal, Glutaraldehyde, Pentanedial, Glutaric aldehyde, Heptenal, cis or trans-Heptenal, Undecenal (2-, 10-), 2,4-octadienal, Nonenal (2-, 6-), Decenal (2-, 4-), 2,4-hexadienal, 2,4-Decadienal, 2,6-Nonadienal, Octenal, 2,6-dimethyl 5-heptenal, 2-isopropyl-5-methyl-2-hexenal, Trifernal, beta methyl Benzenepropanal, 2,6,6-Trimethyl-1-cyclohexene-1-acetaldehyde, phenyl Butenal (2-phenyl 2-butenal), 2.Methyl-3(p-isopropylphenyl)-propionaldehyde, 3-(p-isopropylphenyl)-propionaldehyde, p-Tolylacetaldehyde (4-methylphenylacetaldehyde), Anisaldehyde (p-methoxybenzene aldehyde), Benzaldehyde, Vernaldehyde (1-Methyl-4-(4-methylpentyl)-3-cyclohexenecarbaldehyde), Heliotropin (piperonal) 3,4-Methylene dioxy benzaldehyde, alpha-Amylcinnamic aldehyde, 2-pentyl-3-phenylpropenoic aldehyde, Vanillin (4-methoxy 3-hydroxy benzaldehyde), Ethyl vanillin (3-ethoxy 4-hydroxybenzaldehyde), Hexyl Cinnamic aldehyde, Jasmonal H (alpha-n-hexyl-cinnamaldehyde), Floralozone, (para-ethyl-alpha,alpha-dimethyl Hydrocinnamaldehyde), Acalea (p-methyl-alpha-pentylcinnamaldehyde), methylcinnamaldehyde, alpha-Methylcinnamaldehyde (2-methyl 3-pheny propenal), alpha-hexylcinnamaldehyde (2-hexyl 3-phenyl propenal), Salicylaldehyde (2-hydroxy benzaldehyde), 4-ethyl benzaldehyde, Cuminaldehyde (4-isopropyl benzaldehyde), Ethoxybenzaldehyde, 2,4-dimethylbenzaldehyde, Veratraldehyde (3,4-dimethoxybenz aldehyde), Syringaldehyde (3,5-dimethoxy 4-hydroxybenzaldehyde), Catechaldehyde (3,4-dihydroxybenzaldehyde), Safranal (2,6,6-trimethyl-1,3-diene methanal), Myrtenal (pin-2-ene-1-carbaldehyde), Perillaldehyde L-4(1-methylethenyl)-1-cyclohexene-1-carboxaldehyde), 2,4-Dimethyl-3-cyclohexene carboxaldehyde, 2-Methyl-2-pentenal, 2-methylpentenal, pyruvaldehyde, formyl Tricyclodecan, Mandarin aldehyde, Cyclemax, Pino acetaldehyde, Corps Iris, Maceal, and Corps 4322.

The perfume mixture may include a perfume mixture of one or more perfume raw materials from Table 1.

TABLE 1
Perfume Raw Materials

| CAS # | Name |
|---|---|
| 31375-17-4 | 1-(p-menthen-6(2)-yl)-1-propanone |
| 95962-14-4 | 2-(2-(4-methyl-3-cyclohexen-1-yl)propyl)-cyclopentanone |
| 74338-72-0 | 2,4,4,7-tetramethyl-oct-6-en-3-one |
| 42370-07-0 | 2-acetyl-3,3-dimethyl-norbornane |
| 4433-36-7 | 3,4,5,6-tetrahydropseudoionone |
| 36306-87-3 | 4-(1-ethoxyvinyl)-3,3,5,5,-tetramethyl-cyclohexanone |
| 122-48-5 | 4-(4-hydroxy-3-methoxyphenyl)-2-butanone |
| 4927-36-0 | 4-damascol |
| 37609-25-9 | 5-cyclohexadecenone |
| | 6-isopropyldecahydro-2-naphtone |
| | 8-hexadecenolide |
| 127-41-3 | alpha-ionone |
| 127-42-4 | alpha-Methyl Ionone |
| 119-61-9 | benzophenone |
| 2550-26-7 | benzyl-acetone |
| 75147-23-8 | buccoxime |
| | cassione |
| 3720-16-9 | celery ketone |
| 91462-24-7 | cyclic ethylene dodecanedioate |
| 502-72-7 | cyclopentadecanone |
| 43052-87-5 | damarose alpha |
| 23696-85-7 | Damascenone |
| 2550-11-0 | dimethyl-octenone |
| 55418-52-5 | dulcinyl |
| 105-95-3 | ethylene brassylate |
| 67634-14-4 | floralozone |
| 706-14-9 | Gamma Decalactone |
| 127-51-5 | gamma-methyl ionone |
| 104-50-7 | gamma-Octalactone |
| 108-29-2 | gamma-Valero Lactone |
| 29214-60-6 | gelsone |
| 24851-98-7 | hedione |
| 79-78-7 | hexalon |
| 54464-57-2 | isocyclemone e |
| 70266-48-7 | iso-damascone |
| 54464-57-2 | iso-e-super |
| 24851-98-7 | methy-dihydrojasmonate |
| | methyl beta naphthyl ketone |
| 32388-55-9 | Methyl Cedrylone Major |
| 122-00-9 | methyl-acetophenone |
| | methyl-beta-naphtyl-ketone |
| 32388-55-9 | methyl-cedrenyl-ketone |
| 32388-55-9 | methyl-cedrylone |
| 110-93-0 | methyl-heptenone |
| 67633-95-8 | methyl-lavender-ketone |
| 541-91-3 | muscone |
| 127-43-5 | n-beta-Methyl Ionone Isomer |
| 56973-85-4 | neobutenone |
| 16587-71-6 | orivone |
| 5471-51-2 | Para Hydroxy Phenyl Butanone |
| 100-06-1 | para-methoxy-acetophenone |
| 98-53-3 | para-tert-butyl-cyclohexanone |
| 106-02-5 | pentadecanolide |
| 5471-51-2 | p-Hydroxy Phenyl Butanone |
| 1322-58-3 | tetrameran |
| 21145-77-7 | tonalid |
| 68991-97-9 | 1,2,3,4,5,6,7,8-octahydro-8,8-dimethyl-2-naphthaldehyde |
| 1192-88-7 | 1-cyclohexene-1-carboxaldehyde |
| 66327-54-6 | 1-Methyl-4-(4-methylpentyl)-3-cyclohexenecarbaldehyde |
| 1335-66-6 | 2,4,6-trimethyl-3-cyclohexene-1-carboxaldehyde |
| 25152-84-5 | 2,4-Decadienal |
| 68039-49-6 | 2,4-dimethyl-3-cyclohexen-1-carbaldehyde |
| 68039-49-6 | 2,4-Dimethyl-3-cyclohexene carboxaldehyde |
| 68039-49-6 | 2,4-dimethyl-3-cyclohexene-1-carboxaldehyde |
| 15764-16-6 | 2,4-dimethylbenzaldehyde |
| 68737-61-1 | 2,4-Dimethylcyclohex-3-ene-1-carbaldehyde |
| 142-83-6 | 2,4-hexadienal |
| 30361-28-5 | 2,4-octadienal |
| 24048-13-3 | 2,6,10-trimethyl-5,9-undecadien-1-al |
| 141-13-9 | 2,6,10-Trimethyl-9-undecenal |
| 116-26-7 | 2,6,6-trimethyl-1,3-diene methanal |
| 472-66-2 | 2,6,6-Trimethyl-1-cyclohexene-1-acetaldehyde |
| 106-72-9 | 2,6-dimethyl 5-heptenal |
| 26370-28-5 | 2,6-Nonadienal |
| 103-95-7 | 2.Methyl-3(p-isopropylphenyl)-propionaldehyde |
| 112-54-9 | 2-Dodecanal |
| 613-69-4 | 2-Ethoxybenzaldehyde |
| 97-96-1 | 2-Ethylbutyraldehyde |
| 6728-26-3 | 2-hexenal |
| 101-86-0 | 2-hexyl 3-phenyl propenal |
| 90-02-8 | 2-hydroxy benzaldehyde |
| 35158-25-9 | 2-isopropyl-5-methyl-2-hexenal |
| 101-39-3 | 2-methyl 3-phenyl propenal |
| 96-17-3 | 2-methyl butyraldehyde |
| | 2-methyl deca-1-al |
| 123-15-9 | 2-Methyl Valeraldehyde |
| 110-41-8 | 2-methyl-1-undecanal |
| 623-36-9 | 2-Methyl-2-pentenal |
| 1205-17-0 | 2-Methyl-3-(3,4-methylenedioxyphenyl)propanal |
| | 2-methyl-3-tolylproionaldehyde, 4-dimethylbenzene- |
| | propanal |
| 80-54-6 | 2-methyl-4-t-butylphenyl)propanal |
| 123-15-9 | 2-Methylpentanal |
| 623-36-9 | 2-methylpentenal |
| 122-40-7 | 2-pentyl-3-phenylpropenoic aldehyde |
| 4411-89-6 | 2-phenyl 2-butenal |
| 93-53-8 | 2-phenylproprionaldehyde |
| 125109-85-5 | 3-(3-Isopropyl-phenyl)-butyraldehyde |
| 103-95-7 | 3-(p-isopropylphenyl)-propionaldehyde |
| 139-85-5 | 3,4-dihydroxybenzaldehyde |
| 120-14-9 | 3,4-dimethoxybenzaldehyde |
| 120-57-0 | 3,4-Methylene dioxy benzaldehyde |
| 134-96-3 | 3,5-dimethoxy 4-hydroxybenzaldehyde |
| 106-23-0 | 3,7-dimethyl 6-octenal |
| 107-75-5 | 3,7-dimethyl octan-1-al |
| 106-24-1 | 3,7-dimethyl-2,6-octadien-1-al |
| 7492-67-3 | 3,7-dimethyl-6-octenyl oxyacetaldehyde |
| 121-32-4 | 3-ethoxy 4-hydroxybenzaldehyde |
| 590-86-3 | 3-methyl butyraldehyde |
| 107-86-8 | 3-Methyl-2-butenal |
| 55066-49-4 | 3-methyl-5-phenyl pentanal |
| 16251-77-7 | 3-phenyl butanal |
| 31906-04-4 | 4-(4-hydroxy-4-methyl pentyl)-3-cyclohexene-1- |
| | carboxaldehyde |
| 10031-82-0 | 4-Ethoxybenzaldehyde |
| 4748-78-1 | 4-ethyl benzaldehyde |
| 122-03-2 | 4-isopropyl benzaldehyde |
| 621-59-0 | 4-methoxy 3-hydroxy benzaldehyde |
| 5703-26-4 | 4-methylphenylacetaldehyde |
| 18127-01-0 | 4-t-butylbenzenepropionaldehyde |
| 80-54-6 | 4-tert-butyl-alpha-methyl-hydrocinnamaldehyde |
| | 4-Tricyclo5210-2,6decylidene-8butanal |
| 62439-41-2 | 6-methoxy-2,6-dimethylheptanal |
| 107-75-5 | 7-hydroxy-3,7-dimethyl octan-1-al |
| 84697-09-6 | Acalea |
| 75-07-0 | acetaldehyde |
| 141-13-9 | Adoxal |
| 19009-56-4 | aldehyde C-11 MOA |
| 110-41-8 | aldehyde C12 MNA |
| 122-40-7 | alpha-Amylcinnamic aldehyde |
| 101-86-0 | alpha-hexylcinnamaldehyde |
| 101-39-3 | alpha-Methylcinnamaldehyde |
| 103-95-7 | Alpha-methyl-p-isopropyl phenyl propyl aldehyde |
| 101-86-0 | alpha-n-hexyl-cinnamaldehyde |
| 122-40-7 | Amyl Cinnamic Aldehyde |
| 495-85-2 | amylaldehyde |
| 123-11-5 | Anisic aldehyde |
| 5462-06-6 | anisylpropanal |
| 100-52-7 | Benzaldehyde |
| 104-53-0 | benzenepropanal |
| 65885-41-8 | beta methyl Benzenepropanal |
| 432-25-7 | beta-cyclocitral |
| 18127-01-0 | Bourgeonal |
| 123-72-8 | butyraldehyde |
| 5462-06-6 | canthoxal |
| 139-85-5 | Catechaldehyde |
| 104-55-2 | Cinnamic Aldehyde |
| 6728-31-0 | cis-Heptenal |
| 5392-40-5 | citral |
| 106-23-0 | citronellal |
| 107-75-5 | citronellal hydrate |
| 7492-67-3 | citronellyl oxyacetaldehyde |
| | Corps 4322 |
| | Corps Iris |
| 122-03-2 | Cuminaldehyde |
| 68039-49-6 | cyclal C |
| 103-95-7 | cyclamen aldehyde |
| 7775-00-0 | Cyclemax |
| 68738-96-5 | cyclemone A |
| | cyclocitral |
| 31906-04-4 | cyclohexenyl-carboxaldehyde |
| 103-95-7 | Cymal |
| 112-31-2 | Decenal |
| 5988-91-0 | Dihydrocitronellal |
| 30168-23-1 | duplical |
| 75-07-0 | ethanal |
| 121-32-4 | Ethyl Vanillin |
| 97-53-0 | Eugenol |
| 71077-31-1 | Floral super |
| 67634-14-4 | Floralozone |
| 125109-85-5 | florhydral |
| | formyl Tricyclodecan |
| 5392-40-5 | geranial |
| 111-30-8 | Glutaraldehyde |
| 111-30-8 | Glutaric aldehyde |
| 1205-17-0 | Helional (3-(1,3-Benzodioxol-5-yl)-2-methylpropanal) |
| 120-57-0 | Heliotropin |
| 111-71-7 | heptanal |
| 66-25-1 | Hexenal |
| 101-86-0 | Hexyl Cinnamic aldehyde |
| 90-87-9 | Hydrotropaldehyde |
| 107-75-5 | hydroxycitronellal |
| 1337-83-3 | Intreleven aldehyde |
| 1335-66-6 | Iso Cyclocitral |
| 78-84-2 | isobutyraldehyde |
| 1335-66-6 | iso-Cyclo Citral |
| 590-86-3 | isovaleraldehyde |
| 101-86-0 | Jasmonal H |
| 41496-43-9 | jasmorange |
| 2111-75-3 | L-4(1-methylethenyl)-1-cyclohexene-1-carboxaldehyde |
| 112-54-9 | lauric aldehyde |
| 68039-49-6 | Ligustral |
| 62518-65-4 | Lilestralis 33 |
| 80-54-6 | Lilial |
| | lime aldehyde |
| 51414-25-6 | lyral |
| 80-54-6 | lysmeral |
| 67845-30-1 | Maceal |
| 20407-84-5 | Mandarinal |
| 62518-65-4 | Mefloral |
| 55066-49-4 | mefranal |
| 68991-97-9 | melafleur |
| 106-72-9 | Melonal |
| 62439-41-2 | methoxy melonal |
| | methoxycinnamaldehyde |
| 93-16-3 | Methyl isoeugenol |
| 110-41-8 | methyl nonyl acetaldehyde |
| 19009-56-4 | methyl octyl acetylaldehyde |
| 96-17-3 | methylbutyraldehyde |
| 101-39-3 | methylcinnamaldehyde |
| | Methylthiobutanal |
| 7492-67-3 | muget aldehyde 50 |
| 37677-14-8 | myrac aldehyde |
| 564-94-3 | Myrtenal |
| 173445-65-3 | Neo hivernal |
| 106-26-3 | neral |
| 124-19-6 | nonanal |
| 18829-56-6 | Nonenal |
| 86803-90-9 | octahydro-5-methoxy-4,7-Methano-1H-indene-2- |
| | carboxaldehyde |
| 124-13-0 | octanal |
| 2548-87-0 | Octenal |
| | onicidal |
| 80-54-6 | P.T. Bucinal |
| 67634-14-4 | para-ethyl-alpha,alpha-dimethyl Hydrocinnamaldehyde |
| 110-62-3 | pentanal |
| 111-30-8 | Pentanedial |
| 2111-75-3 | Perillaldehyde |
| 4411-89-6 | phenyl Butenal |
| 14371-10-9 | phenyl propenal, 3-phenyl-2-propenal |
| 122-78-1 | phenylacetaldehyde |
| 564-94-3 | pin-2-ene-1-carbaldehyde |
| 33885-51-7 | Pino acetaldehyde |
| 123-11-5 | p-methoxybenzene aldehyde |
| 101-39-3 | p-methyl-alpha-pentylcinnamaldehyde |
| 52474-60-9 | Precyclemeone B |
| 123-38-6 | propanal |
| 123-38-6 | propionaldehyde |
| 104-09-6 | p-Tolylacetaldehyde |
| 78-98-8 | pyruvaldehyde |
| 116-26-7 | Safranal |
| 90-02-8 | Salicylaldehyde |
| 41496-43-9 | satinaldehyde |
| 86803-90-9 | scentenal |
| 104-09-6 | Syringaldehyde |
| 21944-98-9 | Tangerinal |
| 24680-50-0 | trans-4-methoxycinnamaldehyde |
| 18829-55-5 | trans-Heptenal |
| 30168-23-1 | tricyclodecylidenebutanal |
| 10486-19-8 | tridecanal |
| 16251-77-7 | Trifernal |
| 68039-49-6 | triplal |
| 67801-65-4 | Triplal extra |
| 27939-60-2 | trivertal |
| | undec-10-en-1-al |
| 112-44-7 | Undecenal |
| 110-62-3 | valeraldehyde |
| 121-33-5 | Vanillin |
| 20665-85-4 | Vanillin isobutyrate |
| 120-14-9 | Veratraldehyde |
| 66327-54-6 | Vernaldehyde |
| 68039-49-6 | vertocitral |
| 472-66-2 | β-Homocyclocitral |

The perfume mixture may include perfume raw materials selected from the group consisting of: 3-(1,3-Benzodioxol-5-yl)-2-methylpropanal (trade name Helional), canthoxal, ionone beta, indole, vanillin, ethyl vanillin, cinnamic aldehyde, maceal, triplal extra, 2,6-nonadien-1-al, ligustral, citronellal, eugenol, methyl anthranilate, dimethyl anthranilate and combinations thereof. The perfume mixture may include perfume raw materials selected from the group consisting of: 3-(1,3-Benzodioxol-5-yl)-2-methylpropanal, canthoxal, vanillin, ethyl vanillin, citral, ligustral, cinnamic aldehyde, and combinations thereof. The freshening composition may include from about 0.0001 wt % to about 5 wt %, alternatively from about 0.01 wt % to about 2 wt %, alternatively from about 0.1 wt % to 1.5 wt %, alternatively from about 0.2 wt % to about 1.4 wt %, of perfume raw materials selected from the group consisting of: 3-(1,3-Benzodioxol-5-yl)-2-methylpropanal, canthoxal, vanillin, ethyl vanillin, citral, ligustral, cinnamic aldehyde, and combinations thereof, by weight of the overall composition. The freshening composition may include from about 0.001 wt. % to about 0.5 wt. % of perfume raw materials selected from the group consisting of: 3-(1,3-Benzodioxol-5-yl)-2-methylpropanal, canthoxal, vanillin, ethyl vanillin, citral, ligustral, cinnamic aldehyde, and combinations thereof, by weight of the overall composition. The perfume mixture may comprise dimethyl anthranilate.

The total perfume mixture may be present at an amount from about 0.0001 wt % to about 10 wt %, alternatively from about 0.01 wt % to about 5 wt %, alternatively from about 0.01 wt % to about 2 wt %, alternatively from about 0.1 wt % to 1.5 wt %, alternatively from about 0.2 wt % to about 1.4 wt %, by weight of the overall composition.

The weight ratio of perfume mixture to sulfur-containing pro-perfume may be about 6:1 to about 50:1, or about 6:1 to about 35:1, or about 8:1 to about 25:1, or about 10:1 to about 20:1, by weight of the composition.

The weight ratio of perfume mixture to thio-damascone may be about 6:1 to about 50:1, or about 6:1 to about 35:1, or about 8:1 to about 25:1, or about 10:1 to about 20:1, by weight of the composition.

Without being bound by theory, it is believed that the ratio of perfume mixture to sulfur containing pro-perfume provides a minimum threshold for noticeability of freshness in the air.

### Polyols

The freshening composition may comprise polyols. Low molecular weight polyols with relatively high boiling points, as compared to water, such as ethylene glycol, diethylene glycol, triethylene glycol, propylene glycol, dipropylene glycol, and/or glycerine may be utilized as a malodor counteractant for improving odor neutralization of the freshening composition. Some polyols, e.g., dipropylene glycol, are also useful to facilitate the solubilization of some perfume ingredients in the composition.

The glycol used in the freshening composition may be glycerine, ethylene glycol, propylene glycol, dipropylene glycol, polyethylene glycol, propylene glycol methyl ether, propylene glycol phenyl ether, propylene glycol methyl ether acetate, dipropylene glycol methyl ether acetate, propylene glycol n-butyl ether, dipropylene glycol n-butyl ether, dipropylene glycol n-propyl ether, ethylene glycole phenyl ether, diethylene glycol n-butyl ether, dipropylene glycol n-butyl ether, diethylene glycol mono butyl ether, dipropylene glycol methyl ether, tripropylene glycol methyl ether, tripropylene glycol n-butyl ether, other glycol ethers, or mixtures thereof. The glycol used may be ethylene glycol, propylene glycol, or mixtures thereof. The glycol used may be diethylene glycol.

Typically, the low molecular weight polyol is added to the composition at a level of from about 0.01% to about 5%, by weight of the composition, alternatively from about 0.05% to about 1%, alternatively from about 0.1% to about 0.5%, by weight of the composition. Compositions with higher concentrations may make fabrics susceptible to soiling and/or leave unacceptable visible stains on fabrics as the solution evaporates off of the fabric. The weight ratio of low molecular weight polyol to the malodor binding polymer is from about 500:1 to about 4:1, alternatively from about 1:100 to about 25:1, alternatively from about 1:50 to about 4:1, alternatively about 4:1.

### Cyclodextrin

The freshening composition may include solubilized, water-soluble, uncomplexed cyclodextrin. As used herein, the term “cyclodextrin” includes any of the known cyclodextrins such as unsubstituted cyclodextrins containing from six to twelve glucose units, especially alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms, therefore this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

Cyclodextrin molecules are described in U.S. Pat. No. 5,714,137, and U.S. Pat. No. 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the freshening composition. Freshening compositions with higher concentrations can make fabrics susceptible to soiling and/or leave unacceptable visible stains on fabrics as the solution evaporates off of the fabric. The latter is especially a problem on thin, colored, synthetic fabrics. In order to avoid or minimize the occurrence of fabric staining, the fabric may be treated at a level of less than about 5 mg of cyclodextrin per mg of fabric, alternatively less than about 2 mg of cyclodextrin per mg of fabric.

### Buffering Agent

The freshening composition may include a buffering agent. The buffering agent may be an acidic buffering agent. The buffering agent may be a dibasic acid, carboxylic acid, or a dicarboxylic acid. The carboxylic acid may be, for example, citric acid, polyacrylic acid, or maleic acid. The acid may be sterically stable. The acid may be used in the composition for maintaining the desired pH. The freshening composition may have a pH from about 4 to about 9, alternatively from about 4 to about 8, alternatively from about 4 to about 6.9, alternatively about 4 to about 6.5

Other suitable buffering agents for freshening compositions include biological buffering agents. Some examples are nitrogen-containing materials, sulfonic acid buffers like 3-(N-morpholino)propanesulfonic acid (MOPS) or N-(2-Acetamido)-2-aminoethanesulfonic acid (ACES), which have a near neutral 6.2 to 7.5 pKa and provide adequate buffering capacity at a neutral pH. Other examples are amino acids such as lysine or lower alcohol amines like mono-, di-, and tri-ethanolamine or methyldiethanolamine or derivatives thereof. Other nitrogen-containing buffering agents are tri(hydroxymethyl)amino methane (HOCH2)3CNH3 (TRIS), 2-amino-2-ethyl-1,3-propanediol, 2-amino-2-methyl-propanol, 2-amino-2-methyl-1,3-propanol, disodium glutamate, N-methyl diethanolamide, 2-dimethylamino-2-methylpropanol (DMAMP), 1,3-bis(methylamine)-cyclohexane, 1,3-diamino-propanol N,N′-tetra-methyl-1,3-diamino-2-propanol, N,N-bis(2-hydroxyethyl)glycine (bicine) and N-tris (hydroxymethyl)methyl glycine (tricine). Mixtures of any of the above are also acceptable.

Preferably, freshening compositions include a secondary or tertiary amine. If a secondary or tertiary amine is present, the freshening composition may have a weight ratio of sulfur-containing pro-perfume to secondary or tertiary amine of about 1:1, alternatively the weight of pro-perfume should be equal or higher than the weight of the amine, based on the total weight of the composition. If a secondary or tertiary amine is present, the weight ratio of acidic buffering agent to secondary or tertiary amine may be equal to or greater than 3:1, or greater than 5:1, or greater than 6:1.

The freshening composition may be free of primary amines. Without being bound to theory, it is believed that primary amines inhibit the sulfur-containing pro-perfume reaction with the unstable perfume raw materials.

The freshening compositions may contain at least about 0%, alternatively at least about 0.001%, alternatively at least about 0.01%, by weight of the composition, of a buffering agent. The composition may also contain no more than about 1%, alternatively no more than about 0.75%, alternatively no more than about 0.5%, by weight of the composition, of a buffering agent.

### Solubilizer

The freshening composition may contain a solubilizing aid to solubilize any excess hydrophobic organic materials, particularly any PRMs, and also optional ingredients (e.g., insect repelling agent, antioxidant, etc.) which can be added to the composition, that are not readily soluble in the composition, to form a clear solution. A suitable solubilizing aid is a surfactant, such as a no-foaming or low-foaming surfactant. Suitable surfactants are anionic surfactants, nonionic surfactants, cationic surfactants, amphoteric surfactants, zwitterionic surfactants, and mixtures thereof.

The freshening composition may contain nonionic surfactants, cationic surfactants, and mixtures thereof. The freshening composition may contain surfactant derivatives of hydrogenated castor oil. Suitable ethoxylated hydrogenated castor oils that may be used in the present composition include BASOPHOR™, available from BASF, and CREMOPHOR™, available from Sigma Aldrich.

When the solubilizing agent is present, it is typically present at a level of from about 0.01% to about 3%, alternatively from about 0.05% to about 1%, alternatively from about 0.01% to about 0.05%, by weight of the freshening composition. The freshening composition may have at least as much solubilizer as sulfur-containing pro-perfume to solubilize the pro-perfume into the freshening composition. As such, the freshening composition may comprise a weight ratio of solubilizer to sulfur-containing pro-perfume in the range of about 1:1 or at least the same weight percentage of solubilizer as sulfur-containing pro-perfume, by weight of the composition.

### Antimicrobial Compounds

The freshening composition may include an effective amount of a compound for reducing microbes in the air or on inanimate surfaces. Antimicrobial compounds are effective on gram negative and gram positive bacteria and fungi typically found on indoor surfaces that have contacted human skin or pets such as couches, pillows, pet bedding, and carpets. Such microbial species include Klebsiella pneumoniae, Staphylococcus aureus, Aspergillus niger, Klebsiella pneumoniae, Steptococcus pyogenes, Salmonella choleraesuis, Escherichia coli, Trichophyton mentagrophytes, and Pseudomonoas aeruginosa. The antimicrobial compounds may also effective on viruses such H1-N1, Rhinovirus, Respiratory Syncytial, Poliovirus Type 1, Rotavirus, Influenza A, Herpes simplex types 1 & 2, Hepatitis A, and Human Coronavirus.

Antimicrobial compounds suitable in the freshening composition can be any organic material which will not cause damage to fabric appearance (e.g., discoloration, coloration such as yellowing, bleaching). Water-soluble antimicrobial compounds include organic sulfur compounds, halogenated compounds, cyclic organic nitrogen compounds, low molecular weight aldehydes, quaternary compounds, dehydroacetic acid, phenyl and phenoxy compounds, or mixtures thereof.

A quaternary compound may be used. Examples of commercially available quaternary compounds suitable for use in the freshening composition is BARQUAT® available from Lonza Corporation; and didecyl dimethyl ammonium chloride quat under the trade name BARDAC® 2250 from Lonza Corporation.

The antimicrobial compound may be present in an amount from about 500 ppm to about 7000 ppm, alternatively from about 1000 ppm to about 5000 ppm, alternatively from about 1000 ppm to about 3000 ppm, alternatively from about 1400 ppm to about 2500 ppm, by weight of the freshening composition.

### Preservatives

The freshening composition may include a preservative. The preservative may be included in an amount sufficient to prevent spoilage or prevent growth of inadvertently added microorganisms for a specific period of time, but not sufficient enough to contribute to the odor neutralizing performance of the freshening composition. In other words, the preservative is not being used as the antimicrobial compound to kill microorganisms on the surface onto which the composition is deposited in order to eliminate odors produced by microorganisms. Instead, it is being used to prevent spoilage of the freshening composition in order to increase the shelf-life of the composition.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diaol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% freshening solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name German® II from Sutton Laboratories, Inc.; N,N″-methylenebis{N′-[1-(hydroxymethyl)-2,5-dioxo-4-imidazolidinyl]urea}, commonly known as imidazolidinyl urea, available, e.g., under the trade name Abiol® from 3V-Sigma; Unicide U-13® from Induchem; Germall 115® from Sutton Laboratories, Inc.; polymethoxy bicyclic oxazolidine, available under the trade name Nuosept® C from Hills America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation.

Suitable levels of preservative are from about 0.0001% to about 0.5%, alternatively from about 0.0002% to about 0.2%, alternatively from about 0.0003% to about 0.1%, by weight of the freshening composition.

### Wetting Agent

The freshening composition may, optionally, include a wetting agent that provides a low surface tension that permits the composition to spread readily and more uniformly on hydrophobic surfaces like polyester and nylon. It has been found that the freshening composition, without such a wetting agent will not spread satisfactorily. The spreading of the composition also allows it to dry faster, so that the treated material is ready to use sooner. Furthermore, a composition containing a wetting agent may penetrate hydrophobic, oily soil better for improved malodor neutralization. A composition containing a wetting agent may also provide improved “in-wear” electrostatic control. For concentrated compositions, the wetting agent facilitates the dispersion of many actives such as antimicrobial actives and perfumes in the concentrated freshening compositions.

Non-limiting examples of wetting agents include block copolymers of ethylene oxide and propylene oxide. Suitable block polyoxyethylene-polyoxypropylene polymeric surfactants include those based on ethylene glycol, propylene glycol, glycerol, trimethylolpropane and ethylenediamine as the initial reactive hydrogen compound. Polymeric compounds made from a sequential ethoxylation and propoxylation of initial compounds with a single reactive hydrogen atom, such as C₁₂₋₁₈ aliphatic alcohols, are not generally compatible with the cyclodextrin. Certain of the block polymer surfactant compounds designated Pluronic® and Tetronic® by the BASF-Wyandotte Corp., Wyandotte, Mich., are readily available.

Non-limiting examples of cyclodextrin-compatible wetting agents of this type are described in U.S. Pat. No. 5,714,137 and include the Silwet® surfactants available from Momentive Performance Chemical, Albany, N.Y. Exemplary Silwet® surfactants are as follows:

| Name | Average MW |
|---|---|
| L-7608 | 600 |
| L-7607 | 1,000 |
| L-77 | 600 |
| L-7605 | 6,000 |
| L-7604 | 4,000 |
| L-7600 | 4,000 |
| L-7657 | 5,000 |
| L-7602 | 3,000; |

and mixtures thereof.

The total amount of surfactants (e.g. solubilizer, wetting agent) in the freshening composition is from 0% to about 3% or no more than 3%, alternatively from 0% to about 1% or no more than 1%, alternatively from 0% to about 0.9% or no more than 0.9%, alternatively from 0% to about 0.7 or no more than 0.7%, alternatively from 0% to about 0.5% or no more than 0.5%, alternatively from 0% to 0.3% or no more than about 0.3%, by weight of the composition. Compositions with higher concentrations can make fabrics susceptible to soiling and/or leave unacceptable visible stains on fabrics as the solution evaporates.

The weight ratio of sulfur-containing pro-perfume to total surfactant may be from about 1:1 to about 1:60, or from about 1:1 to about 1:30.

### Carrier

The aqueous composition includes a carrier. The carrier which is used may be water. The water may be distilled, deionized, tap, or further purified forms of water. Water may be present in any amount for the composition to be an aqueous solution. Water may be present in an amount from about 85% to 99.5%, alternatively from about 90% to about 99.5%, alternatively from about 92% to about 99.5%, alternatively from about 95%, by weight of said freshening composition. Water containing a small amount of low molecular weight monohydric alcohols (e.g., ethanol, methanol, and isopropanol, or polyols, such as ethylene glycol and propylene glycol) can also be useful. However, the volatile low molecular weight monohydric alcohols such as ethanol and/or isopropanol should be limited since these volatile organic compounds will contribute both to flammability problems and environmental pollution problems. If small amounts of low molecular weight monohydric alcohols are present in the composition due to the addition of these alcohols to such things as perfumes and as stabilizers for some preservatives, the level of monohydric alcohol may be less than about 6%, alternatively less than about 3%, alternatively less than about 1%, by weight of the freshening composition.

Adjuvants can be optionally added to the freshening composition herein for their known purposes. Such adjuvants include, but are not limited to, water soluble metallic salts, antistatic agents, insect and moth repelling agents, colorants, antioxidants, and mixtures thereof.

### Method of Making

The freshening composition can be made in any suitable manner known in the art. All of the ingredients can simply be mixed together. The method of making may include making a concentrated mixture of ingredients and diluting the concentrated mixture by adding the same to a carrier before dispersing the composition into the air or on an inanimate surface. The method of making may include mixing all of the ingredients except for the sulfur-containing pro-perfume and subsequently adding the sulfur-containing pro-perfume to the mixture of other ingredients. The method may also include pre-mixing the sulfur-containing pro-perfume with some ingredients, such as the solubilizer, polyol, and/or buffering agent before mixing with the other ingredients of the composition.

### Freshening Product

The freshening composition can be packaged in any suitable package to form a freshening product. The package may be in the form of a spray dispenser.

The spray dispenser may be transparent or translucent such that the freshening composition is visible or at least partially visible from outside of the freshening product.

The spray dispenser may be comprised of various materials, including plastic, metal, glass, or combinations thereof. The spray dispenser may be pressurized or unpressurized.

One suitable spray dispenser is a plastic aerosol dispenser. The dispenser may be constructed of polyethylene such as a high density polyethylene; polypropylene; polyethyleneterephthalate (“PET”); vinyl acetate, rubber elastomer, and combinations thereof. The spray dispenser may be made of clear PET.

Another suitable spray dispenser includes a continuous action sprayer, such as FLAIROSOL™ dispenser from Afa Dispensing Group. The FLAIROSOL™ dispenser includes a bag-in-bag or bag-in-can container with a pre-compression spray engine, and aerosol-like pressurization of the freshening composition.

The spray dispenser may hold various amounts of freshening composition.

The spray dispenser may be capable of withstanding internal pressure in the range of about 20 p.s.i.g. to about 140 psig, alternatively about 80 to about 130 p.s.i.g.

The total composition output and the spray droplet/particle size distribution may be selected to support the particulate removal efficacy but avoid a surface wetness problem. Total output is determined by the flow rate of the composition it is released from the spray dispenser. To achieve a spray profile that produces minimal surface wetness, it is desirable to have a low flow rate and small spray droplets.

Flow rate is determined by measuring the rate of composition expelled by a container for any 60 seconds period of use. The flow rate of the composition being released from the spray dispenser may be from about 0.0001 grams/second (g/s) to about 2.5 grams/second. Alternatively, the flow rate may be from about 0.001 grams/second to about 1.8 grams/second, or about 0.01 grams/second to about 1.6 grams/second.

The mean particle size of the spray droplets may be in the range of from about 10 μm to about 100 μm, alternatively from about 20 μm to about 60 μm. At least some of the spray droplets are sufficiently small in size to be suspended in the air for at least about 10 minutes, and in some cases, for at least about 15 minutes, or at least about 30 minutes.

Small particles can be efficiently created when the spray is dispensed in a wide cone angle. For a given nozzle component and delivery tube, cone angles can be modified by varying the insertion depth of the nozzle in the delivery tube. The cone angle may be greater than about 20 degrees, or greater than about 30 degrees, or greater than about 35 degrees, or greater than about 40 degrees, or greater than about 50 degrees.

The spray dispenser may be configured to spray the composition at an angle that is between an angle that is parallel to the base of the container and an angle that is perpendicular thereto. The desired size of spray droplets can be delivered by other types of spray dispensers that are capable of being set to provide a narrow range of droplet size. Such other spray dispensers include, but are not limited to: foggers, ultrasonic nebulizers, electrostatic sprayers, and spinning disk sprayers.

A pressurized spray dispenser may include a propellant. Various propellants may be used. The propellant may comprise a hydrocarbon(s); compressed gas(es), such as nitrogen, carbon dioxide, air; liquefied gas(es) or hydrofluoro olefin (“HFO”); and mixtures thereof. Preferably, the product comprises a propellant selected from the group consisting of compressed gas such as compressed air, compressed nitrogen, and combinations thereof. Propellants listed in the U.S. Federal Register 49 C.F.R. §1.73.115, Class 2, Division 2.2 are considered acceptable. The propellant may particularly comprise a trans-1,3,3,3-tetrafluoroprop-1-ene, and optionally a CAS number 1645-83-6 gas. Such propellants provide the benefit that they are not flammable, although the freshening compositions are not limited to inflammable propellants. One such propellant is commercially available from Honeywell International of Morristown, N.J. under the trade name HFO-1234ze or GWP-6.

If desired, the propellant may be condensable. By “condensable”, it is meant that the propellant transforms from a gaseous state of matter to a liquid state of matter in the spray dispenser and under the pressures encountered in use. Generally, the highest pressure occurs after the spray dispenser is charged with a freshening composition but before that first dispensing of that freshening composition by the user. A condensable propellant provides the benefit of a flatter depressurization curve as the freshening composition is depleted during usage.

The pressurized spray dispenser may be free of a hydrocarbon propellant.

The freshening composition may be delivered from the spray dispenser which includes delivery components including but not limited to a valve to control flow and to seal the freshening composition within the spray dispenser, a button actuator and a nozzle for dispensing the freshening composition to the environment.

The aqueous composition may be contained in a bag-in-can plastic spray dispenser.

### Methods of Use

The freshening composition can be used by dispersing, e.g., by placing the freshening composition into a dispenser, such as a spray dispenser and spraying an effective amount into the air or onto the desired inanimate surface or article. “Effective amount”, when used in connection with the amount of the freshening composition, means an amount sufficient to provide at least about 4 hours, or at least about 6 hours, or at least about 8 hours, or at least about 24 hours of freshness or scent to the treated air, surface, or article, yet not so much as to saturate or create a pool of liquid on an article or surface and so that, when dry, there is no visual deposit readily discernible. Where malodor reducing ingredients are included, “effective amount”, when used in connection with the amount of the freshening composition, means an amount that provides the foregoing and also provides neutralization of a malodor to the point that it is not discernible by the human sense of smell, yet not so much as to saturate or create a pool of liquid on an article or surface and so that, when dry, there is no visual deposit readily discernible. Dispersing can be achieved by using a spray device, a roller, a pad, etc.

All percentages stated herein are by weight unless otherwise specified. It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A freshening product comprising a spray dispenser, the spray dispenser containing a freshening composition and a propellant, the freshening composition comprising:
about 0.02 wt. % to about 1.0 wt. %, based on the weight of the composition, of a sulfur-containing pro-perfume;
a perfume mixture comprising at least one perfume raw material; and
a carrier,
wherein the freshening composition comprises a weight ratio of perfume mixture to sulfur-containing pro-perfume of about 6:1 to about 35:1, by weight of the freshening composition.

2. The freshening product of claim 1, wherein the freshening composition comprises a pH in the range of about 3 to about 9.

3. The freshening product of claim 1, wherein the perfume mixture is present at a level of about 0.2 wt. % to about 1.4 wt. %, by weight of the freshening composition.

4. The freshening product of claim 1, wherein the freshening composition further comprises a carboxylic acid.

5. The freshening product of claim 1, wherein the freshening composition comprises a polyol.

6. The freshening product of claim 1, wherein the perfume mixture comprises at least one material selected from the group consisting of: 3-(1,3-Benzodioxol-5-yl)-2-methylpropanal, canthoxal, vanillin, ethyl vanillin, beta ionone, dimethyl anthranilate, citral, and combinations thereof.

7. The freshening product of claim 1, wherein the sulfur-containing pro-perfume is a C4-C12 thio-damascone.

8. The freshening product of claim 1, wherein the spray dispenser is plastic.

9. The freshening product of claim 1, wherein the propellant comprises compressed gas or a hydrocarbon.

10. The freshening product of claim 1 further comprising a solubilizer.

11. The freshening product of claim 1 further comprising a wetting agent.

12. The freshening product of claim 1, wherein the freshening composition further comprises a secondary or tertiary amine.

13. The freshening product of claim 1, wherein the freshening composition is an aqueous composition.

14. A method of freshening the air, the method comprising the step of:
providing a freshening composition in a pressurized spray dispenser, the freshening composition comprising:
about 0.02 wt. % to about 1.0 wt. %, based on the weight of the composition, of a sulfur-containing pro-perfume;
a perfume mixture comprising at least one perfume raw material; and
a carrier,
wherein the composition comprises a ratio of perfume mixture to sulfur-containing pro-perfume of about 6:1 to about 35:1; and
spraying the freshening composition into the air in the form of spray droplets.

15. The method of claim 14, wherein the composition comprises a pH in the range of about 3 to about 9.

16. The method of claim 14 further comprising a carboxylic acid.

17. The method of claim 14, wherein the perfume mixture comprises at least one material selected from the group consisting of: 3-(1,3-Benzodioxol-5-yl)-2-methylpropanal, canthoxal, vanillin, ethyl vanillin, beta ionone, dimethyl anthranilate, citral, and combinations thereof.

18. The method of claim 14, wherein the sulfur-containing pro-perfume is a C4-C12 thio-damascone.

19. The method of claim 14, wherein the spray droplets have a mean particle size from about 10 microns to about 100 microns.

20. The method of claim 14, wherein the step of spraying the freshening composition further comprises spraying the freshening composition at a flow rate in the range of about 0.1 g/s to about 2.5 g/s.
